(19) United States
(12) Patent Application Publication
(10) Pub. No.: US 2010/0145021 A1
(43) Pub. Date: Jun. 10, 2010

(54) PROCESS FOR ENHANCING PROTEIN RECOVERY YIELDS

(76) Inventor: Wolfgang Marguerre, Heidelberg (DE)

Correspondence Address: JACOBSON HOLMAN PLLC, 400 SEVENTH STREET N.W., SUITE 600, WASHINGTON, DC, 20004, US

(21) Appl. No.: 12/448,844

(22) PCT Filed: Jan. 25, 2008
(86) PCT No.: PCT/EP2008/050898

§ 371 Date: Jan. 22, 2010

(30) Foreign Application Priority Data
Jan. 25, 2007 (EP) 07101200.9

(51) Int. Cl. C07K 14/75; C07K 14/745; C07K 14/755; C07K 16/00; C07K 14/79; C07K 14/47; C07K 14/475; C07K 1/30; C07K 1/00

(52) U.S. Cl. 530/382; 530/380; 530/383; 530/384; 530/387.1; 530/394; 530/395; 530/399; 530/418; 530/421; 530/427

(57) ABSTRACT

A process for enhancing the recovery yield of proteins, especially plasma proteins, from sources containing the proteins, wherein the sources containing the proteins are frozen at temperatures of ≦−70° C., and the proteins from a frozen source after thawing are further processed in a per se known manner.

The present invention relates to a process for enhancing the recovery yield of proteins, especially plasma proteins, from sources containing the proteins.

Sources containing proteins, especially plasma proteins, are a valuable raw material for the recovery of essential factors administered in the form on concentrates to patients with congenital or acquired deficiencies. Suitable concentrates of these factors or combinations of these proteins are employed for the therapy and prophylaxis of various indications. As examples, there may be mentioned concentrates of coagulation factor VIII and of factor IX, which are employed for the treatment of hemophilia A and B, respectively, fractions containing von Willebrand factor are employed for the treatment of the so-called von Willebrand disease, and fibrinogen and prothrombin complex concentrates are employed for the congenital and acquired deficiency of individual factors and combined deficiencies of the corresponding factors. Inhibitors, such as alpha-1 antitrypsin, antithrombin III or C1 esterase inhibitor in the form of concentrates are life-saving medicaments because they limit proteolytic reactions and regulate systems important to hemostasis. In addition, they may also mediate anti-inflammatory and other regulatory functions. Albumin also serves important functions in the maintenance of the blood plasma system and is an essential component of regulation due to its functions of transporting physiological substances and binding toxic ones. Immunoglobulin concentrates are also administered for the prophylaxis and treatment of various inflammatory and dysregulated immunological reactions through the substitution of hereditary or acquired deficiencies. This is usually achieved by intravenous, subcutaneous or intramuscular administration.

In addition to these exemplary plasma components, which are essentially concentrates of different purity, other individual components and their combinations are already being employed or under investigation. These include concentrates of individual or combined factors, such as proteins used for blood clotting in non-activated or activated form, proteins for promoting wound healing, immunoglobulin concentrates of defined specificity, classes (IgG, IgA, IgM, IgE) or subclasses, or modifications prepared therefrom etc.

Plasma is a source of various valuable and life-saving components. The fractionation of plasma and recovery of individual concentrates or combinations of essentially proteins is performed by processes known to the skilled person, such as the so-called process according to Cohn or Kistler-Nitschmann, which results in the enrichment of particular plasma components by the variation and optimization of temperature, pH and ethanol concentration if a corresponding separation is performed by means of precipitation and separation by filtration, centrifugation or other suitable measures. The purpose of such precipitation under defined conditions is an enrichment of one or more plasma components, wherein a complete separation and purification from other plasma components often is not fully achieved by such a step alone. Accordingly, mainly the target components, such as immunoglobulins, are dissolved again by adding suitable solutions, optionally followed by further process steps leading to the final concentrate. These steps mostly consist of further selective precipitations, filtrations and/or chromatographic processes. Part of the preparation process includes steps for the inactivation and separation of potentially infectious components, such as viruses and infectious prions. The skilled person knows, for example, inactivations by the so-called solvent detergent method (EP-A-131 740), pasteurization, treatment with inactivating substances, followed by separation, incubation at acidic pH, UVC irradiation, nanofiltration or other selective processes.

The preparation of the concentrates mostly begins with the combining or “pooling” of individual plasma donations. The individual frozen donations, usually hundreds to many thousands per batch, are combined and thawed under defined conditions to obtain the so-called cryoprecipitate, which contains the coagulation factors FVIII and von Willebrand factor in enriched form, but may also serve as a source for the recovery of fibrinogen and other plasma proteins, such as fibronectin.

After the cryoprecipitate has been separated, the so-called cryo-poor plasma mostly remains as a starting material for the preparation of the other concentrates. The above mentioned process for the separation of main fractions according to Cohn (cryoprecipitate, fraction I, fractions II+III, fractions I+II+III, fraction II, fractions I+III, fraction III, fraction IV, fraction V) and Kistler-Nitschmann (cryoprecipitate, fractions I, IV, precipitates A, B, C, D, G(G)) enriches the target components, such as fibrinogen, factor XIII, immunoglobulins, albumin, alpha-1 antitrypsin and others, and other inhibitors, such as antithrombin III, in particular embodiments in the different precipitates, which can be obtained as described above.

Alternatively or in addition to the precipitations, adsorption are performed with methods and matrices known to the skilled person, including mostly so-called chromatographic gels or special filters, which may have different properties in order to do justice to the specific characteristics of the target proteins and to enable as effective a recovery as possible. For example, coagulation factor IX or prothrombin complex factors (FII, FVII, FIX, FX and additionally the proteins C, S and Z) are typically recovered from the cryo-poor plasma without previous precipitation using gels that have anion-exchange properties. Antithrombin III can be effectively enriched by adsorption on immobilized heparin, often after other plasma components were previously precipitated. However, alternative methods also recover antithrombin III from specific precipitates.

From the cryoprecipitate, fibrinogen, FVIII, vWF or the combination factor VIII/vWF can be recovered by further purifying the components present in the dissolved cryoprecipitate, basically with the above mentioned process steps. However, cryoprecipitate is also employed as such in dissolved form, an optimum recovery yield of the required factors and their integrity being of great importance in this case too.

As set forth above, blood plasma, for example, is a source of many medicaments, mostly in the form of concentrates of especially enriched factors or proteins. The effective utilization of this valuable starting material accordingly requires the separation into the different main fractions and the further processing thereof into the corresponding end products. Due to the high number of the above mentioned factors and possible products from a plasma pool, the exact planning and performance of different methods, often in parallel, are required. Accordingly, a high extent of expenditure, organization and infrastructure is necessary. If such a further processing in parallel of all intermediates obtained into the respective end products is not possible, different preparation intermediates must be appropriately stored. Frequently, equivalent intermediates of different batches are combined in order to optimize the subsequent process steps and the effectiveness.

Since in many cases the intermediates are ones that can be stored only for a certain time as precipitate or in solution without causing damage to the protein, the precipitates or liquid intermediates are frozen and stored in this state. Depending on the starting volume of the plasma pool, these are often very large volumes or precipitate masses. Accordingly, the solutions are often frozen and stored under conditions which are rarely below −30° C., among others for technical reasons.

The so-called “shock freezing” of plasma in liquid nitrogen was described as being advantageous because it was supposed to optimize especially the recovery of coagulation factor VIII as compared to conventional methods. A more effective yield of FVIII was achieved upon reconstitution of the cryoprecipitate obtained. In contrast, the freezing of intermediates, such as the cryoprecipitate itself, or other intermediates (even from shock-frozen plasma) within the scope of product preparation at very low temperatures has not been disclosed.

Without being bound by theory, a possible reason for this can be seen in the fact that the “warming” of solutions and precipitates frozen at very low temperatures for the storage to temperature ranges of below −70° C. and during the thawing process is considered rather disadvantageous, since a restructuring of the crystalline forms of the frozen product can occur in such temperature transitions, which could have rather disadvantageous effects on the nativity and activities of (a) product component(s) and thus could also have a reducing effect on product yields.

The optimization of existing production processes in terms of enhancing the recovery yield of the target proteins while the high quality is maintained is a problem whose solution is the object of the present invention.

The object of the invention is achieved by a process for enhancing the recovery yield of proteins, especially plasma proteins, from sources containing the proteins, especially the plasma proteins, wherein the sources containing the proteins are frozen at temperatures of ≦−70° C. or <−70° C., and the proteins from a frozen source after thawing are further processed in a per se known manner.

Surprisingly, it has been found that the freezing of protein-containing fractions, such as protein precipitates and protein-containing solutions, at temperatures of ≦−70° C. or <−70° C. results in an increase of recovery yield in the thawed intermediates and the resulting final products.

In one embodiment of the process according to the invention, the sources containing proteins, for example, plasma proteins, are frozen by means of liquid nitrogen.

According to the invention, the plasma proteins are selected from the group consisting of immunoglobulins of all classes and subtypes, coagulation factors, other proteins involved in clotting or fibrinolysis, albumin and substances promoting wound closure or wound healing as well as proteins having a transport function.

The immunoglobulins may be IgG, IgM, IgA, IgE and their subclasses.

According to the invention, the recovery yield of the coagulation factors II, V, VII, VIII, IX, X, XI, XII, XIII, fibrinogen and von Willebrand factor can be increased.

According to the invention, the other proteins involved in clotting or fibrinolysis are, in particular, plasminogen, factor VII activating protease, protease inhibitors, such as alpha-1 antitrypsin, antithrombin III or C1-esterase inhibitor, and alpha-2 antiplasmin.

The substances promoting wound closure or wound healing are, in particular, fibronectin, growth factors, such as HGF, FGF, or PDGF.

As proteins having a transport function that can be recovered at an improved yield according to the invention, there may be mentioned transferrin, factors of the complement system or histidine-rich glycoprotein.

In one embodiment, cryoprecipitate, Cohn fractions I, II, III, I+III, II+III, I+II+III, IV, V and combinations thereof or Kistler-Nitschmann fractions I, IV, precipitates A, B, C, D, G(G) and their combinations and modifications are selected as the sources containing plasma proteins for the process according to the invention.

The sources containing proteins, especially plasma proteins, that can be employed according to the invention may be obtainable, for example, from protein precipitates that will denature the plasma proteins to be prepared to less than 50%, especially 30% or 10%.

The protein precipitates may be obtainable by adding polyethylene glycol to the sources containing the proteins, especially the plasma proteins, and/or by salting out proteins from the sources containing the proteins.

In another embodiment of the process according to the invention, the sources containing proteins, especially plasma proteins, frozen at temperatures of ≦−70° C. or <−70° C. can be stored at temperatures of ≦−18° C., especially at ≦−70° C. or <−70° C.

The process according to the invention has proven useful, in particular, for the preparation of immunoglobulins, wherein the starting materials employed for obtaining the immunoglobulins were frozen at temperatures of ≦−70° C. or <−70° C., and their storage was also at temperatures of ≦−70° C. or <−70° C.

Cryoprecipitate is also advantageously frozen and/or stored at temperatures of ≦−70° C. or <−70° C.

Typically, solutions containing plasma proteins, pastes, intermediates from fractions derived from Cohn fractioning or Kistler-Nitschmann fractioning are frozen. Protein-containing fractions from precipitates and solutions derived from recombinant or transgenic preparation may also be frozen and stored. The storage may be effected for a period of at least 12 hours.

According to the process according to the invention, the sources containing the proteins, especially the plasma proteins, may be in the form of protein precipitates that are obtained in different ways. These include methods familiar to the skilled person known for the precipitation according to Cohn and Kistler-Nitschmann (KN), but also by polyethylene precipitation or other methods of precipitation that will not denature the majority of the target proteins, such as precipitation by means of ammonium sulfate or other methods of salting out.

The precipitates from Cohn fractioning are fractions familiar to the skilled person: cryoprecipitate, fraction I, fraction II, fractions II+III, fractions I+II+III, fractions I+III, fraction III, fraction IV and fraction V. The skilled person is also familiar with the fractions according to Kistler-Nitschmann, such as cryoprecipitates A, B and C.

Particularly preferred is the recovery of immunoglobulins by the process according to the invention from fractions I+II+III or II+III and finally fraction II according to Cohn, and from precipitate A according to KN. Alternatively or additionally to the fractions mentioned, precipitates containing immunoglobulins and prepared by polyethylene precipitation, salting out or other precipitation methods may be used.

The invention is further illustrated by the following Example.

### EXAMPLE 1

Fractions I+II+III were obtained by the Cohn method as familiar to the skilled person and further processed into fraction II according to Cohn, which contains the majority of immunoglobulin of G (IgG).

This fraction II was divided into equal parts, which were then treated in different ways:

1. freezing: ≦−25° C.; storage: ≦−25° C.

2. freezing: liquid nitrogen; storage: ≦−70° C.

After storage over a period of three months under the above mentioned conditions, the fractions were reconstituted by the identical method, i.e., the immunoglobulins were essentially dissolved in identical volumes by the Cohn method as known to the skilled person and then filtrated to remove poorly soluble components.

### Result:

The IgG-containing solutions obtained by reconstitution of the various stored precipitates showed no significant differences in product quality when analyzed. However, in batch 2, a significantly higher yield of IgG was achieved. The increase of recovery yield was up to 20% of that achieved when batch 1 was carried out.

### EXAMPLE 2

Cryoprecipitate from pooled frozen individual plasma donations was obtained in the known manner. Aliquots of the precipitate were frozen in liquid nitrogen or at −70° C., and both aliquots were stored at −70° C. After thawing and reconstituting the precipitates (n=4), on average 10% more FVIII activity was found in the samples that had been frozen in liquid nitrogen.

1. A process for enhancing the recovery yield of proteins, especially plasma proteins, from sources containing the proteins, especially the plasma proteins, wherein the sources containing the proteins are frozen at temperatures of ≦−70° C., and the proteins from a frozen source after thawing are further processed in a per se known manner.

2. The process according to claim 1, wherein said sources containing the proteins are frozen by means of liquid nitrogen.

3. The process according to claim 1, wherein plasma proteins are selected from the group consisting of immunoglobulins of all classes and subtypes, coagulation factors, other proteins involved in clotting or fibrinolysis, albumin and substances promoting wound closure or wound healing as well as proteins having a transport function.

4. The process according to claim 3, wherein said immunoglobulins are selected from the group consisting of IgG, IgM, IgA, IgE and their subclasses.

5. The process according to claim 3, wherein said coagulation factors are selected from the group consisting of factors II, V, VII, VIII, IX, X, XI, XII, XIII, fibrinogen and von Willebrand factor.

6. The process according to claim 3, wherein said other proteins involved in clotting or fibrinolysis are selected from the group consisting of plasminogen, factor VII activating protease, protease inhibitors, such as alpha-1 antitrypsin, antithrombin III or C1-esterase inhibitor, and alpha-2 antiplasmin.

7. The process according to claim 3, wherein said substances promoting wound closure or wound healing are selected from the group consisting of fibronectin, growth factors, such as HGF, FGF, or PDGF.

8. The process according to claim 3, wherein said proteins having a transport function are selected from the group consisting of transferrin, factors of the complement system or histidine-rich glycoprotein.

9. The process according to claim 1, wherein said sources containing the plasma proteins are selected from the group consisting of cryoprecipitate, Cohn fractions I, II, III, I+III, II+III, I+II+III, IV, V and combinations thereof or Kistler-Nitschmann fractions I, IV, cryoprecipitate, precipitates A, B, C, D, G(G) and their combinations and modifications.

10. The process according to claim 1, wherein said sources containing the plasma proteins are prepared from protein precipitates that will denature the plasma proteins to be prepared to less than 50%.

11. The process according to claim 10, wherein said protein precipitates are obtainable by adding polyethylene glycol to the sources containing the plasma proteins, and/or by salting out proteins from the sources containing the plasma proteins.

12. The process according to claim 1, wherein the sources containing the plasma proteins frozen at temperatures of ≦−70° C. are stored at temperatures of ≦−18° C., especially at ≦−70° C.

13. The process according to claim 1, wherein solutions containing plasma proteins, pastes, intermediates from fractions derived from Cohn fractioning or Kistler-Nitschmann fractioning are frozen.

14. The process according to claim 13, wherein said storage is effected for a period of at least 12 hours.

15. The process according to claim 1, wherein protein-containing fractions from precipitates and solutions derived from recombinant or transgenic preparation are frozen and stored.
